(19) United States
(12) Patent Application Publication
(10) Pub. No.: US 2018/0140361 A1
(43) Pub. Date: May 24, 2018

(54) NAVIGATION SYSTEM FOR SINUPLASTY DEVICE

(71) Applicant: Pradeep K. Sinha, Atlanta, GA (US)

(72) Inventor: Pradeep K. Sinha, Atlanta, GA (US)

(21) Appl. No.: 15/820,911

(22) Filed: Nov. 22, 2017

Related U.S. Application Data
(60) Provisional application No. 62/426,040, filed on Nov. 23, 2016.

(51) Int. Cl. A61B 34/20; A61B 17/24; A61B 34/10

(52) U.S. Cl. CPC A61B 34/20; A61B 17/24; A61B 34/10; A61B 2090/365; A61B 2034/107; A61B 2034/2055; A61B 2034/2065; A61B 2034/105

(57) ABSTRACT

A method of navigation for a medical device, including, but not limited to, a sinuplasty device, within a portion of a patient's body, the method including mapping a route through a three-dimensional model of the portion patient's body using a target location, manipulating the three-dimensional model so the field of view matches a real-time image, determining whether the target location has been reached, comparing the field of view of the three-dimensional model, determining whether the field of view includes route and non-route passageways through the patient's body, and highlighting and displaying these passageways with different visual markers.

### REFERENCE TO RELATED APPLICATIONS

This application claims the benefit of U.S. Provisional Application No. 62/426,040, filed Nov. 23, 2016 and entitled Navigation System for Sinuplasty Device, the content of which is hereby incorporated by reference in its entirety.

### FIELD OF THE INVENTION

This application relates to navigation systems, and more particularly, to navigation systems for minimally invasive devices, systems, and methods for treating sinusitis and other ear, nose, and throat conditions.

### BACKGROUND

The bones in the nose contain a series of cavities known as paranasal sinuses. Referring to FIGS. 1A and 1B, paranasal sinuses are air-filled spaces in a human's head 10 that surround the nasal cavity 12. The paranasal sinuses include maxillary sinuses 14, which are located under the eyes 16, frontal sinuses 18, which are located above the eyes 16, ethmoidal sinuses 20, which are located between the eyes 16, and sphenoidal sinuses 22, which are located behind the eyes 16. These sinuses are lined with mucous-producing epithelial tissue and ultimately opening into the nasal cavity 12. In normal conditions, mucous produced by the epithelial tissue slowly drains out of each sinus through an opening, which is called an ostium, and into the nasal cavity 12. However, these ostia sometimes can become blocked due to infection, allergies, air pollution, structural problems of the nose, or various other factors that inflame the tissue or otherwise block the passageways. As one example, sinusitis is a condition where the paranasal sinuses are inflamed or infected due to bacteria, viruses, fungi, allergies, or various combinations of factors. Blockage can be acute (resulting in episodes of pain) or chronic.

While it is desirable to treat these blocked passages, treatment of these sinuses is complicated because each sinus presents its own set of challenges for gaining access to the sinus. For example, a surgeon trying to gain access to the frontal sinus 18 must navigate a thin passageway that includes many bends and turns over a relatively long distance (from a medical perspective) before the frontal sinus 18 is reached. Moreover, because the frontal sinuses 18 are proximate to the eyes 16 and the brain, a misstep during navigation, such as excess force applied to a passageway wall, has the potential to result in great harm to the patient. As another example, the surgeon trying to gain access to the sphenoid sinus 22 must almost blindly navigate the passageway to the sphenoid sinus 22 due to the location within the head 10. Moreover, navigation to the sphenoid sinus 22 is further complicated because the sphenoid sinus 22 is near the carotid artery and the skull base of the brain. As such, any missteps during navigation, such as excess force applied to areas of the passageway that cause puncture of the carotid artery, will result in great harm to the patient. As a further example, the surgeon trying to gain access to the maxillary sinus 14, must navigate a small and thin passageway that includes a 135 degree turn to access the maxillary sinus 14. Therefore, because each sinus presents its own set of challenges for gaining access, currently available individual sinuplasty devices are ineffective for treating all of the maxillary sinuses 14, the frontal sinuses 18, and the sphenoidal sinuses 22 as a single device.

### SUMMARY

The terms “invention,” “the invention,” “this invention” and “the present invention” used in this patent are intended to refer broadly to all of the subject matter of this patent and the patent claims below. Statements containing these terms should be understood not to limit the subject matter described herein or to limit the meaning or scope of the patent claims below. Embodiments of the invention covered by this patent are defined by the claims below, not this summary. This summary is a high-level overview of various embodiments of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification of this patent, any or all drawings, and each claim.

According to certain examples, a method of navigation for a medical device within a portion of a body includes: acquiring image data of the portion of the body through an initial image receiver; generating a three-dimensional model of the portion of the body based on the image data using a three-dimensional model generator; receiving at least one target location within the body through a user interface; mapping a route through the three-dimensional model to the at least one target location; acquiring a real-time image and a position and an orientation of the medical device within the body through a real-time image receiver and a position receiver; displaying the real-time image and the three-dimensional model; manipulating the three-dimensional model such that a field of view of the three-dimensional model matches a field of view of the real-time image; determining whether the medical device has reached the at least one target location; determining whether another at least one target location has been provided by the user when the medical device has reached the at least one target location; comparing the field of view of the three-dimensional model to the route when the medical device has not reached the at least one target location; determining whether the field of view of the three-dimensional model includes a passageway that makes up at least a part of the route; highlighting and displaying the passageway within the field of view of the three-dimensional model using a first visual indicator when the passageway that makes up at least a part of the route is within the field of view of the three-dimensional model; determining whether a non-route passageway is within the field of view of the three-dimensional model; and highlighting and displaying the non-route passageway within the field of view of the three-dimensional model using a second visual indicator.

In some examples, the method includes obtaining the image data through an imaging technique comprising at least one of a computerized tomography image, a magnetic resonance imaging image, and an ultrasound image. In various examples, the method includes receiving a target paranasal sinus within the body. In some aspects, the method includes receiving a starting location within the body through the user interface. In various examples, the method includes mapping the route through the three-dimensional model from the starting location to the target location.

A navigator, in some aspects, obtains the three-dimensional model and maps the route through the three-dimensional model from the starting location to the target location. The real-time image, in various examples, includes a real-time endoscopic view. In some examples, the medical device includes a sinuplasty device. In some aspects, the method includes acquiring the real-time image through a lens of a fiber optic probe in communication with the real-time image receiver. In various examples, the method includes displaying the real-time image and the three-dimensional model on a visual output.

In some examples, the method includes determining whether the medical device has reached the at least one target location includes: analyzing the position and the orientation obtained through the real-time image receiver and the position receiver; using the position and the orientation to find a current location of the medical device within the three-dimensional model; and comparing the current location to the route.

In various aspects, the method includes ending the method if no other at least one target location is provided. In various other aspects, the method includes ending the method if another at least one target location was provided and the at least one target location was reached. In some examples, the method includes activating an indicator once the at least one target location has been reached. The indicator, in various examples, includes at least one of a visual indicator, an audible indicator, and a tactile indicator. The indicator, in some aspects, is located on at least one of the medical device, a monitor, a peripheral, and an input-output device. In some examples, the first visual indicator includes at least one of a first color, a first pattern, a first design, a first three-dimensional filling of the passageway, and a first marking of the passageway. The non-route passageway, in various examples, includes the portion of the body that does not make up the at least the part of the route. In some examples, the method includes manipulating the three-dimensional model such that the field of view of the three-dimensional model matches the field of view of the real-time image when the non-route passageways are not within the field of view of the three-dimensional model. The second visual indicator, in some aspects, includes at least one of a second color, a second pattern, and a second design that is different from the first visual indicator.

According to various examples, a method of guiding a sinuplasty device includes: generating a three-dimensional (3D) model of a portion of a patient's body; mapping a route through the 3D model from a start location to a target location; acquiring real-time information of the sinuplasty device within the patient's body; generating a field of view in the 3D model based on the real-time information; comparing the field of view to the route through the 3D model and determining if a portion of the route is within the field of view; and highlighting the portion of the route in the 3D model with a visual marker if the portion of the route is within the field of view.

In certain examples, the method includes displaying a real-time image of the sinuplasty device concurrently with the field of view in the 3D model. In some cases, the method includes receiving a checkpoint location, where mapping the route through the 3D model to the target location includes mapping a route from the start location to the checkpoint location and from the checkpoint location to the target location. In various aspects, generating the 3D model includes acquiring data representing an image of a portion of the patient's body through an initial image receiver. In certain examples, the target location is a paranasal sinus.

In various cases, generating the field of view in the 3D model includes manipulating the field of view in the 3D model such that a field of view in the 3D model matches a field of view of the sinuplasty device in the patient's body in real time. In various examples, determining if the portion of the route is within the field of view includes: determining if a passageway is in the field of view; determining if the passageway is the portion of the route; and highlighting the passageway with a visual marker in the 3D model if the passageway is a portion of the route and in the field of view. In some examples, the visual marker is a first visual marker, and the method further includes: determining if a non-route passageway is within the field of view; and highlighting the non-route passageway with a second visual marker different from the first visual marker in the 3D model if the non-route passageway is in the field of view. According to some cases, the method includes activating an indicator if the target location is in the field of view. In various aspects, the indicator includes a visual indicator, an audible indicator, or a tactile indicator. In certain cases, acquiring the real-time information includes acquiring a position, orientation, and direction of movement of the sinuplasty device within the patient's body.

According to certain examples, a navigation system includes: a sinuplasty device; a three-dimensional (3D) model generator configured to generate a 3D model of a portion of a patient's body; and a navigator in communication with the sinuplasty device and the 3D model generator, and configured to: receive a target location; map a route from a start location to the target location in the 3D model; generate a field of view in the 3D model based on the real-time information; compare the field of view to the route through the 3D model and determine if a portion of the route is within the field of view; and highlight the portion of the route in the 3D model with a visual marker if the portion of the route is within the field of view.

In various cases, the navigation system includes an initial image receiver in communication with the 3D model generator, where the initial image receiver is configured to acquire data representing an image of the portion of the patient's body, and where the 3D model generator is configured to generate the 3D model based on the data from the initial image receiver. In some examples, the navigator is further configured to receive a checkpoint location between the start location and the target location and map a route from the start location to the checkpoint location and from the checkpoint location to the target location. In some aspects, the navigator is further configured to manipulate the 3D model such that the 3D model shows a virtual representation of a same view shown in a corresponding real-time image from the sinuplasty device within the patient's body.

In some cases, the navigator is further configured to determine if the if the target location is in the field of view and activate an indicator if the target location is in the field of view. In certain aspects, the indicator includes a visual indicator, an audible indicator, or a tactile indicator. In various aspects, the visual marker is a first visual marker, and the navigator is further configured to determine if a non-route passageway is within the field of view and highlight the non-route passageway with a second visual marker different from the first visual marker in the 3D model if the non-route passageway is in the field of view. In certain examples, the visual marker is a color. In some examples, the target location is a paranasal sinus.

Various implementations described in the present disclosure can include additional systems, methods, features, and advantages, which cannot necessarily be expressly disclosed herein but will be apparent to one of ordinary skill in the art upon examination of the following detailed description and accompanying drawings. It is intended that all such systems, methods, features, and advantages be included within the present disclosure and protected by the accompanying claims.

### BRIEF DESCRIPTION OF THE DRAWINGS

The features and components of the following figures are illustrated to emphasize the general principles of the present disclosure. Corresponding features and components throughout the figures can be designated by matching reference characters for the sake of consistency and clarity.

FIG. 1A is a view of the paranasal sinuses within a patient.

FIG. 1B is another view of the paranasal sinuses within the patient.

FIG. 2A is a side view of a sinuplasty device including a fiber optic probe, a guide, and an introducer according to an aspect of the current disclosure.

FIG. 2B is a side view of a sinuplasty device according to an aspect of the current disclosure

FIG. 3 is a diagram of a computer apparatus according to an aspect of the current disclosure.

FIGS. 4A and 4B are a flowchart of an exemplary process performed by the computer apparatus of FIG. 3.

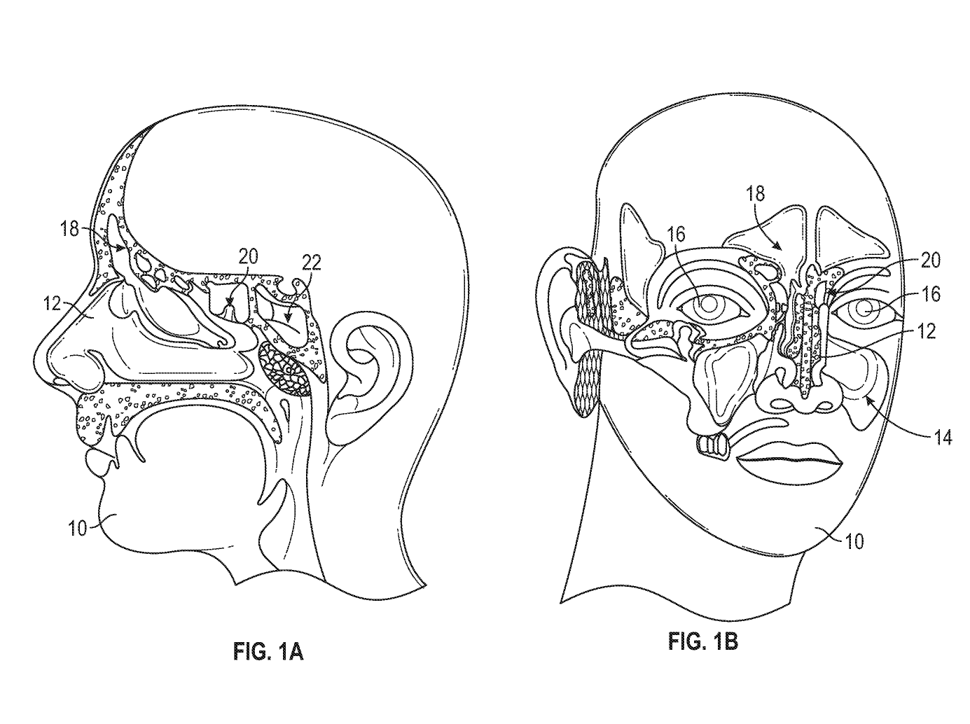

### DETAILED DESCRIPTION

The present invention can be understood more readily by reference to the following detailed description, examples, drawings, and claims, and their previous and following description. However, before the present devices, systems, and/or methods are disclosed and described, it is to be understood that this invention is not limited to the specific devices, systems, and/or methods disclosed unless otherwise specified, and, as such, can, of course, vary. It is also to be understood that the terminology used herein is for describing particular aspects only and is not intended to be limiting.

The following description of the invention is provided as an enabling teaching of the invention in its best, currently known embodiment. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

As used throughout, the singular forms “a,” “an” and “the” include plural referents unless the context clearly dictates otherwise. Thus, for example, reference to “a band” can include two or more such bands unless the context indicates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance can or cannot occur, and that the description includes instances where said event or circumstance occurs and instances where it does not.

The word “or” as used herein means any one member of a particular list and includes any combination of members of that list. Further, one should note that conditional language, such as, among others, “can,” “could,” “might,” or “can,” unless specifically stated otherwise, or otherwise understood within the context as used, is generally intended to convey that certain aspects include, while other aspects do not include, certain features, elements, and/or steps. Thus, such conditional language is not generally intended to imply that features, elements and/or steps are in any way required for one or more particular aspects or that one or more particular aspects necessarily include logic for deciding, with or without user input or prompting, whether these features, elements and/or steps are included or are to be performed in any particular embodiment. Directional references such as “up,” “down,” “top,” “left,” “right,” “front,” “back,” and “corners,” among others are intended to refer to the orientation as illustrated and described in the figure (or figures) to which the components and directions are referencing.

Various implementations described in the present disclosure can include additional systems, methods, features, and advantages, which cannot necessarily be expressly disclosed herein but will be apparent to one of ordinary skill in the art upon examination of the following detailed description and accompanying drawings. It is intended that all such systems, methods, features, and advantages be included within the present disclosure and protected by the accompanying claims.

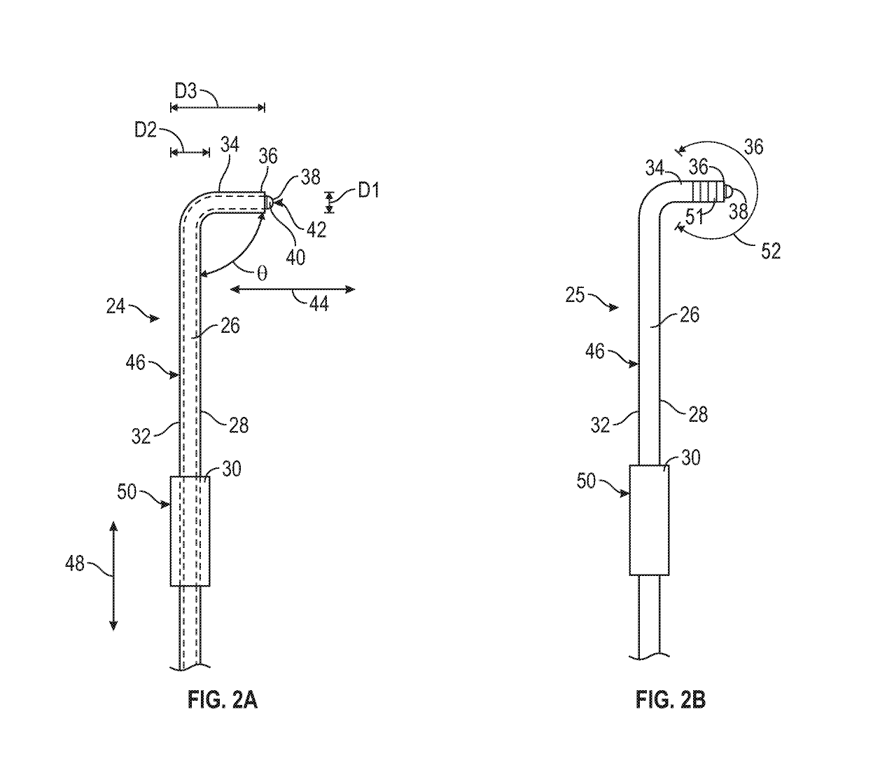

Referring to FIG. 2, in one aspect, disclosed is a sinuplasty device 24 and associated methods, systems, devices, and various apparatus. As described in detail below, the sinuplasty device 24 is a combination visualization and introduction device that can be used to gain access to the paranasal sinuses and introduce various items to the paranasal sinuses. It would be understood by one of skill in the art that the disclosed sinuplasty device 24 is described in but a few exemplary aspects among many.

In various aspects, the sinuplasty device 24 includes a fiber optic probe 26, a guide 28, and an introducer 30. The guide 28 is a housing having an insertion end 36 and a connecting end (not shown). In various aspects, the insertion end 36 is configured to be inserted into the human body, while the connecting end is configured to either engage a holder that can be held by a person using the sinuplasty device 24 or serve as the holder that the person can directly hold. The guide 28 includes main portion 32 between the insertion end 36 and the connecting end, and a tip portion 34 between the main portion 32 and the insertion end 36. As described in detail below, the guide 28 is hollow such that the fiber optic probe 26 is movable through the guide 28.

As illustrated in FIG. 2A, the tip portion 34 is at an angle θ relative to the main portion 32. In some aspects, the angle θ is an angle suitable for gaining access to at least one of the maxillary sinus 14, the frontal sinus 18, or the sphenoidal sinuses 22. In these aspects, the angle θ can be between about 0° and about 135°. For example and without limitation, the angle θ can be 0°, 30°, 45°, 60°, 70°, 90°, 120°, or 135°. In other examples, the tip portion 34 may be movable to angles greater than 135° (see, e.g., FIG. 2B).

In some examples, the tip portion 34 is an articulating or malleable tip portion 34 that is movable to the various angles θ relative to the main portion 32. In this aspect, the angle θ of the tip portion 34 can be adjusted depending on which paranasal sinus is being accessed. In other examples, the tip portion 34 is rigid such that the tip portion 34 is fixed at a predefined angle θ. In these examples, each paranasal sinus may have a dedicated tip portion 34. The shape of each tip portion 34 may determine which tip portion 34 is associated with a particular sinus. For example and without limitation, the sinuplasty device 24 can include a removable fontal sinus tip portion, a removable sphenoidal sinus tip portion, and a removable maxillary sinus tip portion.

The tip portion 34 may also be malleable such that various shapes, designs, or configurations may be formed by the guide 28 as needed. For example and without limitation, in some cases, the tip portion 34 may be formed or shaped to comprise multiple angles.

In some cases, the tip portion 34 may be mechanically bendable or malleable. In these examples, the tip portions 34 may be an extension of a hand piece (not shown) for the guide 28 that the user may grasp. In various cases, the tip portion 34 is mechanically bendable or malleable through various mechanisms including, but not limited to gears, turn-wheels, motors, screws, and various other suitable movement mechanisms. These mechanisms may rotate the tip portion 34 through various angles and shapes to allow for coaxial approach to the target duct.

FIG. 2B illustrates another example of a sinuplasty device 25. The sinuplasty device 25 is substantially similar to the sinuplasty device 24 except that the tip portion 34 includes a number of articulating segments 51. Although four articulating segments 51 are illustrated, any number of articulating segments 51 may be provided including one articulating segment 51, two articulating segments 51, three articulating segments 51, or more than four articulating segments 51. In various examples, the articulating segments 51 are hingedly connected to adjacent articulating segments 51 and/or the probe 26 such that the tip portion 34 may articulate through an angle 52 in opposing directions. In various cases, the articulating segments 51 are constructed from various materials that are capable of bending while retaining some degree of rigidity similar, but not limited, to the materials of a bicycle chain, chainmail, or other similar. In one non-limiting example, the angle 52 is from 0° to about 270°, although various other ranges of the angle 52 may be provided. In some cases, the number of articulating segments 51 may allow for a narrower or wider angle 52 of articulation. As one non-limiting example, fewer articulating segments 51 may allow for a narrower angle 52 and additional segments 51 may allow for a greater angle 52. In some examples, the articulating segments 51 allow for articulation in at least one pair of opposing directions (e.g., articulation up and down or left to right). In other examples, the articulating segments 51 allow for articulating in two pairs of opposing directions (e.g., articulation both up and down and left to right). In various examples, the portion of the device 25 with the articulating segments 51 may be from about 7 mm to about 12 mm, although in other examples, the portion may be less than 7 mm or greater than 12 mm. In some examples, an internal opposing system, similar, but not limited, to a pulley system, would cause the tip portion 34 with articulating segments 51 to bend in opposing directions, such as up/down. In other cases, a second internal opposing system would cause the tip portion 34 and/or guide 28 to bend in perpendicular directions, such as left/right.

Referring back to FIG. 2A, in examples where the tip portions 34 are rigid and at fixed angles θ, the tip portions 34 may be removably connected to the main portion 32 through threading, snap-fitting, or various other suitable mechanisms. In this manner, different tip portions 34 can be connected to and removed from the main portion 32 depending on which paranasal sinus is being accessed. In other examples where the tip portions 34 are at fixed angles θ, the main portion 32 and tip portion 34 are integrally formed as a single component guide 28. In these examples, multiple guides 28 can be utilized with the sinuplasty device 24, and each guide 28 can have the tip portion 34 at a different angle θ such that each guide 28 is dedicated to a different paranasal sinus. In this manner, the guide 28 used with the sinuplasty device 24 can be changed depending on which paranasal sinus is to be accessed.

In these examples where the tip portions 34 are removably connected to the main portion 32 or where different guides 28 dedicated to different paranasal sinuses can be interchangeably used with the sinuplasty device 24, the tip portions 34 and/or main portions 32 can be constructed from various autoclavable materials. For example and without limitation, the autoclavable materials may be various metals such as stainless steel or other comparable metals and alloys, various glass, plastics, or other composite materials, and various other suitable materials. In this aspect, the tip portions 34 can be reusable. In other aspects, the main portion 32 may also be constructed from various autoclavable materials such that the main portion 32 can be reusable. In various examples where the tip portions 34 are not removably connected to the main portion 32 and different shaped guides 28 are provided for each sinus, the entire guide 28 may be autoclavable.

The fiber optic probe 26 is the core of the sinuplasty device 24 and is configured to be moved through the guide 28. As described in detail below, the fiber optic probe 26 is coaxial with the guide 28, and the guide 28 surrounds at least a portion of the fiber optic probe 26. In various aspects, the fiber optic probe 26 includes a light delivery system (not shown) configured to illuminate those spaces into which the fiber optic probe 26 is inserted. The fiber optic probe 26 has a viewing end 38 and a connecting end (not illustrated). In various examples, the viewing end 38 includes a lens 40. The lens 40 is aspherical in various examples such that the lens 40 is configured to have a wide angle view. In these examples, a curved surface 42 of the aspherical lens 40 is configured to reduce the likelihood of traumatic impact by the fiber optic probe 26 as compared to a probe having a flat or angled surface. The connecting end of the fiber optic probe 26 is connected to a visualization output device, such as an eye-piece, monitors, or other suitable devices for outputting the view obtained through the viewing end 38.

The fiber optic probe 26 is flexible and has a thin diameter D1 such that the fiber optic probe 26 can access and navigate the various bends and turns of the passageways to each paranasal sinus. As a non-limiting example, the fiber optic probe 26 may be similar to that used with the product Marchal All-In-One Sialendoscope, sold by Karl Storz GmbH & Co. The Marchal All-In-One Sialendoscope may be similar to those fiber optic probes described in U.S. Pat. No. 9,351,530 or U.S. Pat. No. 7,850,604, the content of which is hereby incorporated by reference in their entireties. Various other suitable fiber optic probes 26 may be utilized. As illustrated in FIG. 2 by the arrow 44, the fiber optic probe 26 is movable through the guide 28 such that the viewing end 38 can be at a position proximate or distal to the insertion end 36 of the guide 28. In some aspects, the viewing end 38 can be movable to within an interior of the guide 28, although it need not be. In some examples, the fiber optic probe 26, the guide 28, or both may include a stopper to limit the extent to which the fiber optic probe 26 moves through the guide 28. This may provide a predetermined length at which the viewing end 28 can be positioned from the insertion end 36. In various cases, the predefined length may be from about 0 mm to about 150 mm. For example and without limitation, the predefined length may be from about 10 mm to about 140 mm, such as from about 20 mm to about 130 mm, such as from about 30 mm to about 120 mm, such as from about 40 mm to about 110 mm, such as from about 50 mm to about 100 mm, such as from about 50 mm to about 90 mm, such as from about 60 mm to about 80 mm. Various other suitable ranges for the predetermined length may be utilized.

The introducer 30 is coaxial with the guide 28 and the fiber optic probe 26 and configured to be movable along the guide 28, as indicated by the arrow 48 in FIG. 2. In this aspect, the introducer 30 is exterior to the guide 28 and is configured to move along at least a portion of an outer surface 46 of the guide 28 between the connecting end of the guide 28 and the insertion end 36 of the guide 28. In some examples, the introducer 30 is manually movable along the guide 28, such as through sliding or other suitable means. In other examples, the introducer 30 may be mechanically moved through various suitable mechanisms such as gears, turn-wheels, motors, screws, and various other suitable movement mechanisms. In various cases, the guide 28 may include a stopper to retain the introducer 30 on the guide 28. In this aspect, the introducer 30 is exterior to the guide 28 and is configured to move along at least a portion of an outer surface 46 of the guide 28 between the connecting end of the guide 28 and the insertion end 36 of the guide 28.

In some examples, the introducer 30 may also be movable along at least a portion of the probe 26 in addition to or in place of being movable along the guide 28. In some cases, the introducer 30 may be movable between the insertion end 36 of the guide 28 and the viewing end 38 of the probe 26. In other cases, the introducer 30 may be movable between the connecting end of the guide 28 and the viewing end 38 of the probe 26, or between various other positions along the guide 28 and/or the probe 26. Similar to the guide 28, in cases where the introducer 30 is movable along the probe 26, the probe 26 may include a stopper that is configured to retain the introducer 30 on the sinuplasty device 24.

The introducer 30 defines an introducing surface 50 onto which a number of items to be introduced into the patient via the sinuplasty device 24 can be embedded, removably attached, or otherwise connected to the introducer 30. As one non-limiting example, a stent to be introduced into the patient is removably attached to the introducer 30. In other examples, a balloon for dilating an opening within the patient, such as the ostium of one of the paranasal sinuses, may be attached. In other examples, the introducer 30 may define locations on the introducing surface 50 where various devices such as a drill, knife, vacuum tube, fluid tube or nozzle, spray tube or nozzle, other nozzles, or other devices to be used within the patient may be attached. In various other examples, the introducer 30 may define locations on the introducing surface 50 where various drugs to be released within the patient may be retained.

In various aspects, the introducer 30 may define a fixed diameter D2. However, in various other examples, the diameter of the introducer 30 is expandable such that the diameter of the introducer 30 can increase to the diameter D3 illustrated in FIG. 2. In these examples, the diameter of the introducer 30 may be expandable through various expanding and contracting mechanisms including, but not limited to, rotating members, dilators, screws, hinges, pushable members, and various other suitable expanding and contracting mechanisms.

In some examples, the fiber optic probe 26, guide 28, and introducer 30 are coaxial. The fiber optic probe 26 is movable within the interior of the guide 28 defined by an inner surface of the guide 28 (not shown). The introducer 30 is movable along the outer surface 46 of the guide 28.

In various examples, some or all of the components of the sinuplasty device 24 may be shaped and have various properties to aid the user using the sinuplasty device 24. For example, in some examples, at least a portion of the guide 28 may provide an ergonomic fit to the user's hand. In these examples, the portion of the guide 28 may be designed for grasping and may have tackiness to aid in grip, may have a shape for aiding in gripping, or may have various other properties or designs. As one non-limiting example, the portion of the guide 28 may have a triangular shape, although it need not.

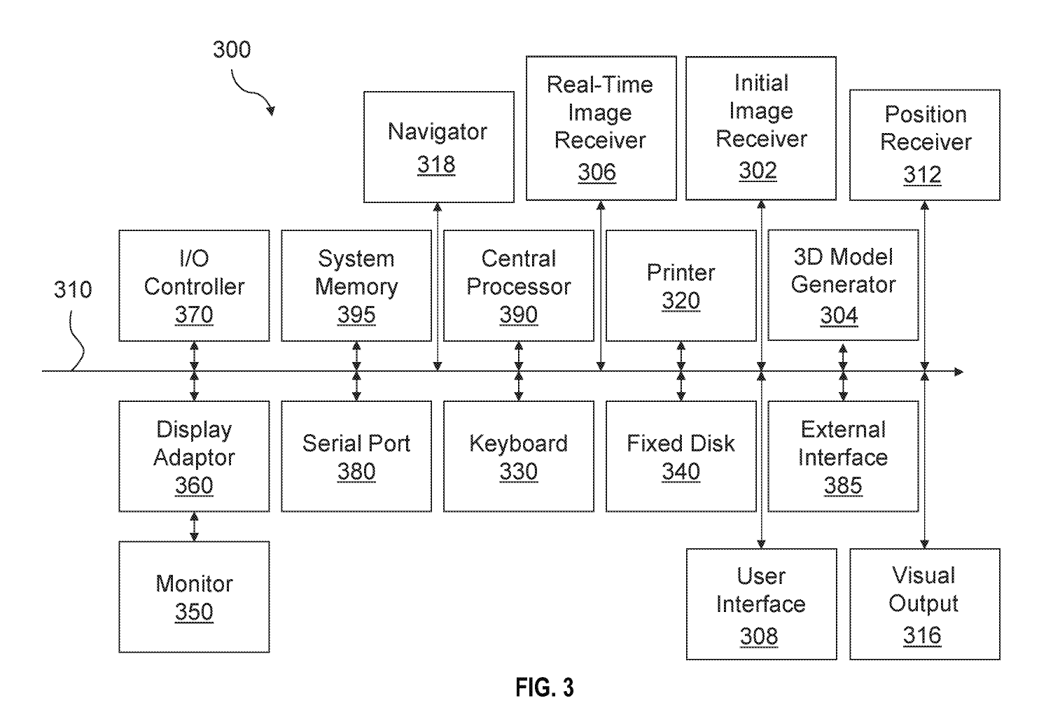

FIG. 3 illustrates a navigation system 300. In some aspects, the navigation system 300 may be used with the sinuplasty device 24; however, in various other examples, the navigation system 300 may be used with various other types of sinuplasty devices or other types of medical devices to be inserted and guided through the patient's body.

The system 300 can facilitate navigation of a medical device to a desired location within a patient's body. As one non-limiting example, the system 300 can facilitate navigation of the sinuplasty device 24 through the patient's body to any one of the paranasal sinuses.

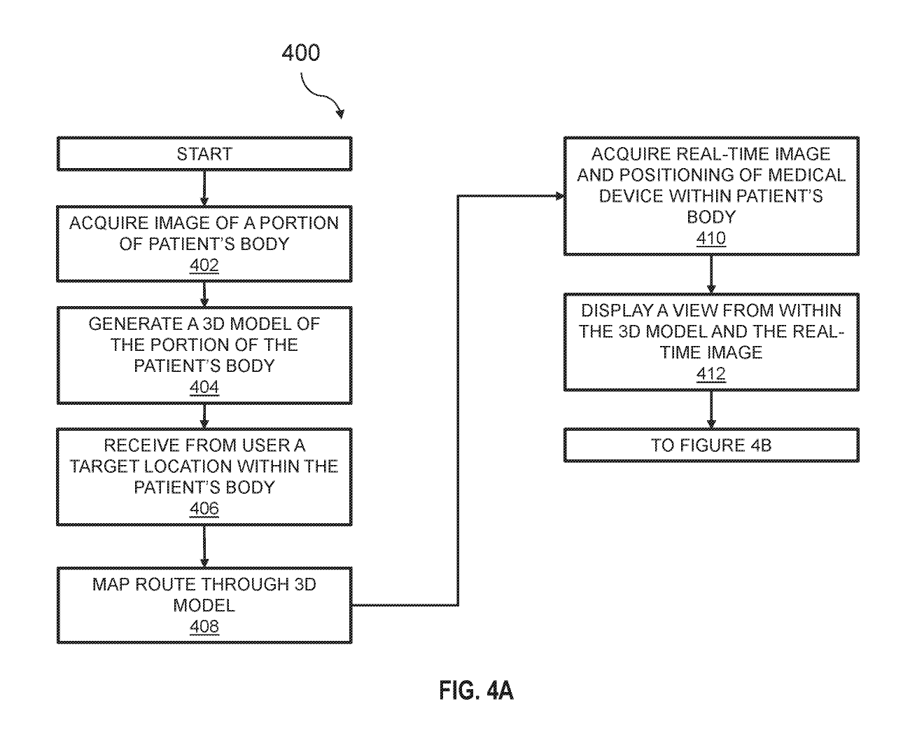

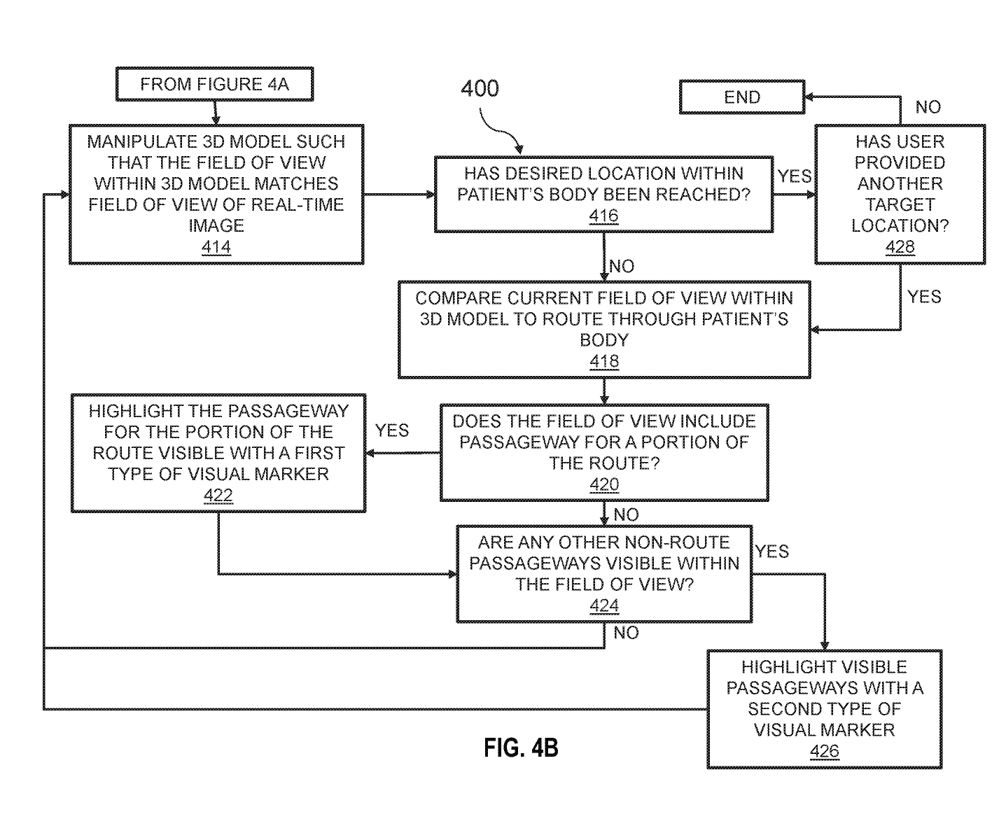

In some examples, the functions and processes of one or more components of the system 300 to facilitate navigation of the medical device, such as the processes illustrated in FIGS. 4A and 4B and described below, can be implemented by computer program instructions.

The functions of one or more of the components can be implemented by computer program instructions. These computer program instructions can be provided to a processor of a general purpose computer, special purpose computer, and/or other programmable data processing apparatus to produce a machine, such that the instructions, which execute via the processor of the computer and/or other programmable data processing apparatus, create a mechanism for implementing the functions of the components specified in the block diagrams. These computer program instructions can also be stored in a non-transitory computer-readable memory that can direct a computer or other programmable data processing apparatus to function in a particular manner, such that the instructions stored in the non-transitory computer-readable memory produce an article of manufacture including instructions, which implement the function specified in the block diagrams and associated description. The computer program instructions can also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions of the components specified in the block diagrams and the associated description.

Accordingly, the system 300 described herein can be embodied at least in part in hardware and/or in software (including firmware, resident software, micro-code, etc.). Furthermore, aspects of the system 300 can take the form of a computer program product on a computer-usable or computer-readable storage medium having computer-usable or computer-readable program code embodied in the medium for use by or in connection with an instruction execution system. A computer-usable or computer-readable medium can be any non-transitory medium that is not a transitory signal and can contain or store the program for use by or in connection with the instruction or execution of a system, apparatus, or device. The computer-usable or computer-readable medium can be, for example but not limited to, an electronic, magnetic, optical, electromagnetic, infrared, or semiconductor system, apparatus or device. More specific examples (a non-exhaustive list) of the computer-readable medium can include the following: a portable computer diskette; a random access memory; a read-only memory; an erasable programmable read-only memory (or Flash memory); and a portable compact disc read-only memory.

FIG. 3 is a diagram of a computer apparatus 300 according to an example embodiment. The various participants and elements may use any suitable number of subsystems in the computer apparatus 300 to facilitate the functions described herein. Examples of such subsystems or components are shown in FIG. 3. The subsystems shown in FIG. 3 may be interconnected via a system bus 310. Additional subsystems such as a printer 320, keyboard 330, fixed disk 340 (or other memory comprising computer-readable media), monitor 350, which is coupled to display adapter 360, and others are shown. In various examples, these and other various user interfaces may be configured to receive inputs from the user, such as a desired or target location within the patient's body and/or a desired route or pathway through the patient's body to the desired location. The user interfaces can also show real-time images from within the patient's body and/or view a 3D model or portions of a 3D model of the patient's body. Various other inputs and outputs may be provided through the user interfaces.

Peripherals and input/output (I/O) devices (not shown), which couple to I/O controller 370, can be connected to the computer system by any number of means known in the art, such as serial port 380. For example, serial port 380 or external interface 385 can be used to connect the computer apparatus 300 to a wide area network such as the Internet, a mouse input device, or a scanner. The interconnection via system bus allows the central processor 390 to communicate with each subsystem and to control the execution of instructions from system memory 395 or the fixed disk 340, as well as the exchange of information between subsystems. The system memory 395 and/or the fixed disk 340 may embody a computer-readable medium.

The software components or functions described in this application may be implemented as software code to be executed by one or more processors using any suitable computer language such as, for example, Java, C++, or Perl using, for example, conventional or object-oriented techniques. The software code may be stored as a series of instructions, or commands on a computer-readable medium, such as a random access memory (RAM), a read-only memory (ROM), a magnetic medium such as a hard-drive or a floppy disk, or an optical medium such as a CD-ROM. Any such computer-readable medium may also reside on or within a single computational apparatus, and may be present on or within different computational apparatuses within a system or network.

The invention can be implemented in the form of control logic in software or hardware or a combination of both. The control logic may be stored in an information storage medium as a plurality of instructions adapted to direct an information-processing device to perform a set of steps disclosed in embodiments of the invention. Based on the disclosure and teachings provided herein, a person of ordinary skill in the art will appreciate other ways and/or methods to implement the invention. In embodiments, any of the entities described herein may be embodied by a computer that performs any or all of the functions and steps disclosed.

In some examples, the memory comprises instructions for an initial image receiver. The initial image receiver is configured to receive data representing an image of a portion of the patient's body. In some cases, the image data is obtained through an imaging techniques including, but not limited to a CT (computerized tomography) image, an MRI (magnetic resonance imaging) image, an ultrasound image, or various other imaging techniques. In some examples, the image data is received prior to a planned medical procedure, although it need not be.

The memory may also comprise instructions for a three dimensional (3D) model generator. The 3D model generator is configured to generate a 3D model of the portion of the patient's body based on the image data acquired by the initial image receiver.

In various examples, the memory may include instructions for a real-time image receiver. The real-time image receiver is configured to receive a real-time image, such as a real-time endoscopic view, of the position and orientation of the medical device within the patient's body. As used herein, images may include still images, moving images such as videos, animations, etc., combinations thereof, and/or any other suitable kinds of images. In some examples where the sinuplasty device 24 is utilized, the connecting end of the fiber optic probe 26 is connected to the real-time image receiver such that the real-time image receiver 306 can acquire a view obtained through the lens 40.

The memory may include instructions for a position receiver, which is configured to receive and track the positioning and movement of the medical device. The memory may further include instructions for a navigator, which is configured to receive the data from the 3D model generator 304, the real-time image receiver 306, the position receiver 312, and the user interface 308 and to manipulate the data for the user as described below to facilitate navigation of the medical device

FIGS. 4A and 4B show a flowchart of exemplary steps of a method 400 that may be taken using the system 300 to provide navigation for a medical device within a portion of the patient's body. In one aspect, in step 402, the method 400 includes acquiring data representing an image of a portion of the patient's body through the initial image receiver 302. As described above, the image data can be obtained through imaging techniques including, but not limited to a CT (computerized tomography) image, an MRI (magnetic resonance imaging) image, an ultrasound image, or various other imaging techniques. In some examples, the image data is received prior to a planned medical procedure, although it need not be.

In another aspect, in a step 404, the method 400 includes generating a 3D model of the portion of the patient's body based on the image data acquired in step 402 through the 3D model generator 304. In a step 406, the method 400 includes receiving, through the user interface 308, a target location within the patient's body. In various cases, the method 400 includes receiving multiple target locations within the patient's body. In some of these cases, the multiple target locations may be utilized as checkpoints to get to a final target location. Step 406 may also include receiving a starting location from the user through the user interface 308. As one non-limiting example, in step 406, the user interface 308 receives a target paranasal sinus, such as the sphenoid sinus 22, that the user would like to reach in the patient's body.

In a step 408, the method 400 includes mapping a route through the 3D model of the portion of the patient's body generated in step 404 to the target location provided in step 406. In some aspects, mapping the route through the 3D model includes mapping the route from the starting location to the target location, both which may be provided in step 406. In some aspects, the navigator 318 obtains the 3D model generated in step 404 and the target location from step 406 and maps the desired route through the 3D model of the patient's body.

In some aspects, in a step 410, the method 400 includes acquiring a real-time image, such as a real-time endoscopic view, and the position and orientation of the medical device within the patient's body through the real-time image receiver 306 and the position receiver 312. As described above, in examples where the system 300 is used with the sinuplasty device 24, acquiring the real-time image in step 410 includes acquiring the image through the lens 40 of the fiber optic probe 26 in communication with the real-time image receiver 306.

In a step 412, the method 400 includes displaying to the user, such as on the visual output 316, the real-time image or endoscopic view from step 410 and the 3D model of the portion of the patient's body from step 404. In some examples, in step 414, the navigator 318 uses the position, view, and orientation data of the real-time image from the real-time image receiver 306 and the position receiver 312 to manipulate the view of the 3D model visually displayed to the user. The navigator 318 manipulates the view of the 3D model such that position, orientation, and direction of movement of the field of view of the 3D model matches that of the real-time image. In other words, the navigator 318 manipulates the 3D model such that the 3D model shows a virtual representation of the same view shown in the real-time image. In some cases, the 3D model may show additional views or other views that are different from that shown in the real-time image. For example and without limitation, in various examples, the 3D model may also show various planar views of the model. In other cases, other real-time views may be provided with the endoscopic and 3D model views, including, but not limited to, various planar views such as CT image scans, or various other images or views.

In a step 416, the navigator 318 determines whether the medical device has reached the desired location within the patient's body. In some examples, the navigator 318 makes this determination by analyzing the position, orientation, and direction of movement data obtained through the real-time image receiver 306 and position receiver 312, using that data to find a current location of the medical device within the 3D model, and comparing the current location within the 3D model to the desired route received in step 406.

If the navigator 318 determines that the medical device has reached the desired location in step 416, in step 428, the process determines whether another target location has been provided by the user. If another target location has been provided by the user, the process proceeds to step 418. In some cases, the process may further determine if the other target location has already been reached. In various cases, if no other target is provided by the user, the process ends. In some cases, if another target location has been provided by the user but the target location has already been reached, the process may also end. In some cases, the process may activate an indicator upon determining that the medical device has reached the desired location. In some examples, the indicator may be a visual indicator, an audible indicator, tactile indicator, or various other suitable indicators. The indicator may be provided on the medical device, the monitor 350, peripherals or other I/O devices, or various other components of the system 300.

If the navigator 318 determines that the medical device has not reached the desired location, in step 418, the method 400 includes the navigator 318 comparing the current field of view (including the position, orientation, and direction of movement) within the 3D model to the desired route through the patient's body.

In step 420, the navigator 318 determines whether the current field of view includes a passageway that makes up at least a portion of the desired route. If the navigator 318 determines that a passageway that makes up at least a portion of the desired route is within the current field of view, the method 400 proceeds to step 422 where the navigator 318 visually highlights the passageway within the current field of view with a first visual indicator and displays the highlighted passageway to the user. In some examples, visually highlighting the passageway with the first visual indicator includes visually highlighting the passageway with a first color, pattern, design, 3D filling of the passageway, marking of the passageway, or various other suitable types of visual markers. The method 400 then proceeds to step 424. If in step 420, the navigator 318 determines that the current field of view does not include a passageway that makes up at least a portion of the desired route, the method proceeds to step 424.

In step 424, the method 400 includes determining, by the navigator 318, whether any other non-route passageways are within the field of view. Non-route passageways include those portions of the patient's body that do not make up at least a portion of the desired route through the patient's body. If, in step 424, the navigator 318 determines that other non-route passageways are within the field of view, the method proceeds to step 426. In step 426, the navigator 318 visually highlights the passageway within the current field of view with a second visual indicator and displays the highlighted passageway to the user. In some examples, visually highlighting the passageway with the second visual indicator includes visually highlighting the passageway with a second color, pattern, design, or various other suitable types of visual markers that is different from the first visual indicator. As one non-limiting example, in some aspects, a passageway that makes up at least a portion of the desired route may be highlighted in a first color, such as the color green, and a non-route passageway may be highlighted in a second color, such as the color red. In this aspect, the user can visually identify the direction of the desired route within the current field of view.

After step 426, the method 400 returns to step 414. In other aspects, if in step 424, the navigator 318 determines that other non-route passageways are not within the field of view, the method 400 returns to step 414.

A collection of exemplary embodiments, including at least some explicitly enumerated as “ECs” (Example Combinations), providing additional description of a variety of embodiment types in accordance with the concepts described herein are provided below. These examples are not meant to be mutually exclusive, exhaustive, or restrictive; and the invention is not limited to these example embodiments but rather encompasses all possible modifications and variations within the scope of the issued claims and their equivalents.

EC 1. A method of navigation for a medical device within a portion of a body, the method comprising: acquiring image data of the portion of the body through an initial image receiver; generating a three-dimensional model of the portion of the body based on the image data using a three-dimensional model generator; receiving at least one target location within the body through a user interface; mapping a route through the three-dimensional model to the at least one target location; acquiring a real-time image and a position and an orientation of the medical device within the body through a real-time image receiver and a position receiver; displaying the real-time image and the three-dimensional model; manipulating the three-dimensional model such that a field of view of the three-dimensional model matches a field of view of the real-time image; determining whether the medical device has reached the at least one target location; determining whether another at least one target location has been provided by the user when the medical device has reached the at least one target location; comparing the field of view of the three-dimensional model to the route when the medical device has not reached the at least one target location; determining whether the field of view of the three-dimensional model includes a passageway that makes up at least a part of the route; highlighting and displaying the passageway within the field of view of the three-dimensional model using a first visual indicator when the passageway that makes up at least a part of the route is within the field of view of the three-dimensional model; determining whether a non-route passageway is within the field of view of the three-dimensional model; and highlighting and displaying the non-route passageway within the field of view of the three-dimensional model using a second visual indicator.

EC 2. The method of any of the preceding or subsequent example combinations, further comprising obtaining the image data through an imaging technique comprising at least one of a computerized tomography image, a magnetic resonance imaging image, and an ultrasound image.

EC 3. The method of any of the preceding or subsequent example combinations, further comprising receiving a target paranasal sinus within the body.

EC 4. The method of any of the preceding or subsequent example combinations, further comprising receiving a starting location within the body through the user interface.

EC 5. The method of any of the preceding or subsequent example combinations, further comprising mapping the route through the three-dimensional model from the starting location to the target location.

EC 6. The method of any of the preceding or subsequent example combinations, wherein a navigator obtains the three-dimensional model and maps the route through the three-dimensional model from the starting location to the target location.

EC 7. The method of any of the preceding or subsequent example combinations, wherein the real-time image comprises a real-time endoscopic view.

EC 8. The method of any of the preceding or subsequent example combinations, wherein the medical device comprises a sinuplasty device.

EC 9. The method of any of the preceding or subsequent example combinations, further comprising acquiring the real-time image through a lens of a fiber optic probe in communication with the real-time image receiver.

EC 10. The method of any of the preceding or subsequent example combinations, further comprising displaying the real-time image and the three-dimensional model on a visual output.

EC 11. The method of any of the preceding or subsequent example combinations, wherein determining whether the medical device has reached the at least one target location comprises: analyzing the position and the orientation obtained through the real-time image receiver and the position receiver; using the position and the orientation to find a current location of the medical device within the three-dimensional model; and comparing the current location to the route.

EC 12. The method of any of the preceding or subsequent example combinations, further comprising ending the method if no other at least one target location is provided.

EC 13. The method of any of the preceding or subsequent example combinations, further comprising ending the method if another at least one target location was provided and the at least one target location was reached.

EC 14. The method of any of the preceding or subsequent example combinations, further comprising activating an indicator once the at least one target location has been reached.

EC 15. The method of any of the preceding or subsequent example combinations, wherein the indicator comprises at least one of a visual indicator, an audible indicator, and a tactile indicator.

EC 16. The method of any of the preceding or subsequent example combinations, wherein the indicator is located on at least one of the medical device, a monitor, a peripheral, and an input-output device.

EC 17. The method of any of the preceding or subsequent example combinations, wherein the first visual indicator comprises at least one of a first color, a first pattern, a first design, a first three-dimensional filling of the passageway, and a first marking of the passageway.

EC 18. The method of any of the preceding or subsequent example combinations, wherein the non-route passageway comprises the portion of the body that does not make up the at least the part of the route.

EC 19. The method of any of the preceding or subsequent example combinations, further comprising manipulating the three-dimensional model such that the field of view of the three-dimensional model matches the field of view of the real-time image when the non-route passageways are not within the field of view of the three-dimensional model.

EC 20. The method of any of the preceding or subsequent example combinations, wherein the second visual indicator comprises at least one of a second color, a second pattern, and a second design that is different from the first visual indicator.

EC 21. A method of guiding a sinuplasty device comprising: generating a three-dimensional (3D) model of a portion of a patient's body; mapping a route through the 3D model from a start location to a target location; acquiring real-time information of the sinuplasty device within the patient's body; generating a field of view in the 3D model based on the real-time information; comparing the field of view to the route through the 3D model and determining if a portion of the route is within the field of view; and highlighting the portion of the route in the 3D model with a visual marker if the portion of the route is within the field of view.

EC 22. The method of any of the preceding or subsequent example combinations, further comprising displaying a real-time image of the sinuplasty device concurrently with the field of view in the 3D model.

EC 23. The method of any of the preceding or subsequent example combinations, further comprising receiving a checkpoint location, wherein mapping the route through the 3D model to the target location comprises mapping a route from the start location to the checkpoint location and from the checkpoint location to the target location.

EC 24. The method of any of the preceding or subsequent example combinations, wherein generating the 3D model comprises acquiring data representing an image of a portion of the patient's body through an initial image receiver.

EC 25. The method of any of the preceding or subsequent example combinations, wherein the target location is a paranasal sinus.

EC 26. The method of any of the preceding or subsequent example combinations, wherein generating the field of view in the 3D model comprises manipulating the field of view in the 3D model such that a field of view in the 3D model matches a field of view of the sinuplasty device in the patient's body in real time.

EC 27. The method of any of the preceding or subsequent example combinations, wherein determining if the portion of the route is within the field of view comprises: determining if a passageway is in the field of view; determining if the passageway is the portion of the route; and highlighting the passageway with a visual marker in the 3D model if the passageway is a portion of the route and in the field of view.

EC 28. The method of any of the preceding or subsequent example combinations, wherein the visual marker is a first visual marker, and wherein the method further comprises: determining if a non-route passageway is within the field of view; and highlighting the non-route passageway with a second visual marker different from the first visual marker in the 3D model if the non-route passageway is in the field of view.

EC 29. The method of any of the preceding or subsequent example combinations, further comprising activating an indicator if the target location is in the field of view.

EC 30. The method of any of the preceding or subsequent example combinations, wherein the indicator comprises a visual indicator, an audible indicator, or a tactile indicator.

EC 31. The method of any of the preceding or subsequent example combinations, wherein acquiring the real-time information comprises acquiring a position, orientation, and direction of movement of the sinuplasty device within the patient's body.

EC 32. A navigation system comprising: a sinuplasty device; a three-dimensional (3D) model generator configured to generate a 3D model of a portion of a patient's body; a navigator in communication with the sinuplasty device and the 3D model generator, and configured to: receive a target location; map a route from a start location to the target location in the 3D model; generate a field of view in the 3D model based on the real-time information; compare the field of view to the route through the 3D model and determine if a portion of the route is within the field of view; and highlight the portion of the route in the 3D model with a visual marker if the portion of the route is within the field of view.

EC 33. The navigation system of any of the preceding or subsequent example combinations, further comprising an initial image receiver in communication with the 3D model generator, wherein the initial image receiver is configured to acquire data representing an image of the portion of the patient's body, and wherein the 3D model generator is configured to generate the 3D model based on the data from the initial image receiver.

EC 34. The navigation system of any of the preceding or subsequent example combinations, wherein the navigator is further configured to receive a checkpoint location between the start location and the target location and map a route from the start location to the checkpoint location and from the checkpoint location to the target location.

EC 35. The navigation system of any of the preceding or subsequent example combinations, wherein the navigator is further configured to manipulate the 3D model such that the 3D model shows a virtual representation of a same view shown in a corresponding real-time image from the sinuplasty device within the patient's body.

EC 36. The navigation system of any of the preceding or subsequent example combinations, wherein the navigator is further configured to determine if the if the target location is in the field of view and activate an indicator if the target location is in the field of view.

EC 37. The navigation system of any of the preceding or subsequent example combinations, wherein the indicator comprises a visual indicator, an audible indicator, or a tactile indicator.

EC 38. The navigation system of any of the preceding or subsequent example combinations, wherein the visual marker is a first visual marker, and wherein the navigator is further configured to determine if a non-route passageway is within the field of view and highlight the non-route passageway with a second visual marker different from the first visual marker in the 3D model if the non-route passageway is in the field of view.

EC 39. The navigation system of any of the preceding or subsequent example combinations, wherein the visual marker is a color.

EC 40. The navigation system of any of the preceding or subsequent example combinations, wherein the target location is a paranasal sinus.

It should be emphasized that the above-described aspects are merely possible examples of implementations, merely set forth for a clear understanding of the principles of the present disclosure. Many variations and modifications can be made to the above-described embodiment(s) without departing substantially from the spirit and principles of the present disclosure. All such modifications and variations are intended to be included herein within the scope of the present disclosure, and all possible claims to individual aspects or combinations of elements or steps are intended to be supported by the present disclosure. Moreover, although specific terms are employed herein, as well as in the claims that follow, they are used only in a generic and descriptive sense, and not for the purposes of limiting the described invention, nor the claims that follow.

That which is claimed:

1. A method of guiding a sinuplasty device comprising:
generating a three-dimensional (3D) model of a portion of a patient's body;
mapping a route through the 3D model from a start location to a target location;
acquiring real-time information of the sinuplasty device within the patient's body;
generating a field of view in the 3D model based on the real-time information;
comparing the field of view to the route through the 3D model and determining if a portion of the route is within the field of view; and
highlighting the portion of the route in the 3D model with a visual marker if the portion of the route is within the field of view.

2. The method of claim 1, further comprising displaying a real-time image of the sinuplasty device concurrently with the field of view in the 3D model.

3. The method of claim 1, further comprising receiving a checkpoint location, wherein mapping the route through the 3D model to the target location comprises mapping a route from the start location to the checkpoint location and from the checkpoint location to the target location.

4. The method of claim 1, wherein generating the 3D model comprises acquiring data representing an image of a portion of the patient's body through an initial image receiver.

5. The method of claim 1, wherein the target location is a paranasal sinus.

6. The method of claim 1, wherein generating the field of view in the 3D model comprises manipulating the field of view in the 3D model such that a field of view in the 3D model matches a field of view of the sinuplasty device in the patient's body in real time.

7. The method of claim 1, wherein determining if the portion of the route is within the field of view comprises:
determining if a passageway is in the field of view;
determining if the passageway is the portion of the route; and
highlighting the passageway with a visual marker in the 3D model if the passageway is a portion of the route and in the field of view.

8. The method of claim 7, wherein the visual marker is a first visual marker, and wherein the method further comprises:
determining if a non-route passageway is within the field of view; and
highlighting the non-route passageway with a second visual marker different from the first visual marker in the 3D model if the non-route passageway is in the field of view.

9. The method of claim 1, further comprising activating an indicator if the target location is in the field of view.

10. The method of claim 9, wherein the indicator comprises a visual indicator, an audible indicator, or a tactile indicator.

11. The method of claim 1, wherein acquiring the real-time information comprises acquiring a position, orientation, and direction of movement of the sinuplasty device within the patient's body.

12. A navigation system comprising:
a sinuplasty device;
a three-dimensional (3D) model generator configured to generate a 3D model of a portion of a patient's body; and
a navigator in communication with the sinuplasty device and the 3D model generator, and configured to:
receive a target location;
map a route from a start location to the target location in the 3D model;
generate a field of view in the 3D model based on the real-time information;
compare the field of view to the route through the 3D model and determine if a portion of the route is within the field of view; and
highlight the portion of the route in the 3D model with a visual marker if the portion of the route is within the field of view.

13. The navigation system of claim 12, further comprising an initial image receiver in communication with the 3D model generator, wherein the initial image receiver is configured to acquire data representing an image of the portion of the patient's body, and wherein the 3D model generator is configured to generate the 3D model based on the data from the initial image receiver.

14. The navigation system of claim 12, wherein the navigator is further configured to receive a checkpoint location between the start location and the target location and map a route from the start location to the checkpoint location and from the checkpoint location to the target location.

15. The navigation system of claim 12, wherein the navigator is further configured to manipulate the 3D model such that the 3D model shows a virtual representation of a same view shown in a corresponding real-time image from the sinuplasty device within the patient's body.

16. The navigation system of claim 12, wherein the navigator is further configured to determine if the if the target location is in the field of view and activate an indicator if the target location is in the field of view.

17. The navigation system of claim 16, wherein the indicator comprises a visual indicator, an audible indicator, or a tactile indicator.

18. The navigation system of claim 12, wherein the visual marker is a first visual marker, and wherein the navigator is further configured to determine if a non-route passageway is within the field of view and highlight the non-route passageway with a second visual marker different from the first visual marker in the 3D model if the non-route passageway is in the field of view.

19. The navigation system of claim 12, wherein the visual marker is a color.

20. The navigation system of claim 12, wherein the target location is a paranasal sinus.
